(19) United States
(12) Patent Application Publication
(10) Pub. No.: US 2025/0291017 A1
(43) Pub. Date: Sep. 18, 2025

(54) Efficient Magnetic Resonance Elastography using Interleaved Motion Encoding

(71) Applicants: Siemens Healthineers AG, Forchheim (DE); Centre National de la Recherche Scientifique (CNRS), Paris Cedex (FR); Institut National de la Sante et de la Recherche Medicale (INSERM), Paris (FR); King's College London, London (GB); UNIV PARIS XIII PARIS-NORD VILLETANEUSE, Villetaneuse (FR); Université Paris Cité, Paris (FR)

(72) Inventors: Donovan Tripp, London (GB); Omar Darwish, London (GB); Ralph Sinkus, Parmain (FR); Radhouene Neji, London (GB)

(73) Assignees: Siemens Healthineers AG, Forchheim (DE); Centre National de la Recherche Scientifique (CNRS), Paris Cedex 16 (FR); Institut National de la Sante et de la Recherche Medicale (INSERM), Paris (FR); King's College London, London (GB); UNIV PARIS XIII PARIS-NORD VILLETANEUSE, Villetaneuse (FR); Université Paris Cité, Paris (FR)

(21) Appl. No.: 19/078,397

(22) Filed: Mar. 13, 2025

(30) Foreign Application Priority Data
Mar. 13, 2024 (EP) 24163235.5

(51) Int. Cl. G01R 33/563; A61B 5/00; A61B 5/055; G01R 33/56

(52) U.S. Cl. CPC G01R 33/56358; A61B 5/0051; A61B 5/055; A61B 5/4244; G01R 33/5608

(57) ABSTRACT

In a method for performing Magnetic Resonance Elastography (MRE) more efficiently may include providing a periodical vibration signal for exciting mechanical vibrations within an object to be examined with a vibration period, sampling the vibration signal with a sampling period corresponding to a natural number including zero of vibration periods plus a fixed time delay, and performing three motion encoding gradients for magnetic resonance acquisition in each sampling period. The fixed time delay multiplied with a sampling number may be equal to the vibration period. The sampling number may be a natural number greater than two.

### CROSS REFERENCE TO RELATED APPLICATIONS

This patent application claims priority to European Patent Application No. 24163235.5, filed Mar. 13, 2024, which is incorporated herein by reference in its entirety.

### BACKGROUND

### Field

The present disclosure relates to a method of performing Magnetic Resonance Elastography. Furthermore, the present disclosure relates to a Magnetic Resonance Elastography device and a computer program for controlling such device.

### Related Art

A fundamental paper of Magnetic Resonance Elastography (MRE) is Muthupillai R, Lomas D J, Rossman P J, Greenleaf J F, Manduca A, Ehman R L. Magnetic resonance elastography by direct visualization of propagating acoustic strain waves. Science. 1995 Sep. 29; 269(5232):1854-7.

MRE enables non-invasive estimation of biomechanical tissue properties in-vivo allowing in-vivo estimation of biomechanical properties in tissues, with applications including the assessment and staging of liver fibrosis as shown in Venkatesh, S. K., Yin, M. and Ehman, R. L. (2013), Magnetic resonance elastography of liver: Technique, analysis, and clinical applications. J. Magn. Reson. Imaging, 37: 544-555.

The simultaneous assessment of liver fibrosis and inflammation from elasticity and viscosity has previously been demonstrated with 3D MRE sequences by Sinkus, R, Lambert, S, Abd-Elmoniem, K Z, et al. Rheological determinants for simultaneous staging of hepatic fibrosis and inflammation in patients with chronic liver disease. NMR in Biomedicine. 2018; 31:e3956 and by Darwish O I, Gharib A M, Jeljeli S, Metwalli N S, Feeley J, Rotman Y, Brown R J, Ouwerkerk R, Kleiner D E, Stab D, Speier P, Sinkus R, Neji R. Single Breath-Hold 3-Dimensional Magnetic Resonance Elastography Depicts Liver Fibrosis and Inflammation in Obese Patients. Invest Radiol. 2023 Jun. 1; 58(6):413-419.

MRE reconstruction using the curl operator to remove the compressional wave was performed by Sinkus, R., Tanter, M., Catheline, S., Lorenzen, J., Kuhl, C., Sondermann, E. and Fink, M. (2005), Imaging anisotropic and viscous properties of breast tissue by magnetic resonance-elastography. Magn. Reson. Med., 53: 372-387.

Current volumetric 3D MRE sequences are typically acquired in 2D slices, missing out on the higher SNR afforded by a 3D acquisition.

Published 3D MRE techniques are based on wave offset interleaving like van Schelt A S, Gottwald L M, Wassenaar N P M, Runge J H, Sinkus R, Stoker J, Nederveen A J, Schrauben E M. Single Breath-Hold MR Elastography for Fast Biomechanical Probing of Pancreatic Stiffness. J Magn Reson Imaging. 2023 May 17. doi: 10.1002/jmri.28773. Epub ahead of print. PMID: 37194646.

A generalized multi-shot MRE sequence is shown by Guenthner C, Sethi S, Troelstra M, Dokumaci A S, Sinkus R, Kozerke S. Ristretto MRE: A generalized multi-shot GRE-MRE sequence. NMR Biomed. 2019 May; 32(5):e4049. doi: 10.1002/nbm.4049. Epub 2019 Jan. 29. PMID: 30697827; PMCID: PMC6590281.

A minimum cost flow technique for unwrapping phase images is presented in Costantini M. A novel phase unwrapping method based on network programming. IEEE Transactions on Geoscience and Remote Sensing. 1998; 36(3):813-821. doi:10.1109/36.673674.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 illustrates Magnetic Resonance Elastography (MRE) device according to one or more exemplary embodiments.

FIG. 2 is a timing diagram for the acquisition of all readouts at one phase encoding with the proposed sequence, according to one or more exemplary embodiments.

FIG. 3A illustrates acquisition results includes a calculated curl of the wave field in the Z direction of the object (e.g. liver) according to one or more exemplary embodiments.

FIG. 3B illustrates acquisition results showing a magnitude of the reference scan without motion encoding.

FIG. 3C illustrates acquisition results showing a total wave amplitude according to one or more exemplary embodiments.

FIG. 3D illustrates acquisition results showing viscoelastic parameters to explain the behavior/state of the tissue including shear wave speed Cs according to one or more exemplary embodiments.

FIG. 3E illustrates acquisition results showing viscoelastic parameters to explain the behavior/state of the tissue including a magnitude of the complex valued shear modulus |G*|, which is correlated with the speed Cs according to one or more exemplary embodiments.

FIG. 3F illustrates acquisition results showing viscoelastic parameters to explain the behavior/state of the tissue including a loss modulus G″ according to one or more exemplary embodiments.

The exemplary embodiments of the present disclosure will be described with reference to the accompanying drawings. Elements, features and components that are identical, functionally identical and have the same effect are—insofar as is not stated otherwise—respectively provided with the same reference character.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure. The connections shown in the figures between functional units or other elements can also be implemented as indirect connections, wherein a connection can be wireless or wired. Functional units can be implemented as hardware, software or a combination of hardware and software.

An object of the present disclosure is to provide an efficient acquisition scheme for Magnetic Resonance Elastography. Specifically, a respective method for performing MRE and a respective MRE device shall be provided.

According to the present disclosure this object is solved by a method and a Magnetic Resonance Elastography device according to one or more exemplary embodiments of the disclosure.

A method of performing Magnetic Resonance Elastography is therefore proposed including the step of providing a periodical vibration signal for exciting mechanical vibrations with a vibration period. This means that elastography based on magnetic resonance tomography is performed. A periodical vibration signal is provided as basis for the elastography. This periodical vibration signal can serve for exciting mechanical vibrations. For instance, an electromechanical transducer can be supplied with a periodical vibration signal.

In a further step the vibration signal is sampled with a sampling period. The sampling period is also called “wave phase” in this document. The sampling period corresponds to a natural number including 0 of vibration periods plus a fixed delay. For instance, the sampling period is equal to 2,25 vibration periods. Thus, the natural number of vibration periods is two and the fixed delay is a quarter of a vibration period. According to another example the sampling period may also be equal to 0,125 vibration periods, which means that the natural number of the vibration period is 0 and the fixed delay is ⅛ of a vibration period. The number of vibration periods within one sampling period can also be significantly larger.

The fixed delay multiplied with a sampling number is equal to the vibration period, wherein the sampling number is a natural number greater than 2. This means that the vibration signal is sampled more than two times in one vibration period. For instance, a vibration signal is sampled 3, 4, 5 etc. times within one vibration period. The fixed delay ensures a certain phase shift of the sampling period with respect to the vibration period. After a number of sampling periods corresponding to the sampling number the phase shift between the vibration period and the sampling period is 0. Thus, the acquisition can be continued with a new sampling period without time delay.

Three motion encoding gradients (and optional a reference scan) are performed for magnetic resonance acquisition in each sampling period. This means that the sampling period, i.e. the wave phase, is divided into four time slots. Three time slots are used for applying motion encoding gradients. In an exemplary embodiment, the gradients are different with respect to spatial directions. A fourth time slot is used for a reference scan. The reference scan can be used to standardize other measurement scans.

The advantage of the inventive method is that a very efficient scheme is provided compared to other known techniques of the prior art.

According to an exemplary embodiment, the motion encoding gradients are part of a 3D slab-selective Magnetic Resonance Imaging sequence. The slab-selective stimulation includes a stimulation within a slice as well as a stimulation perpendicular to the slice.

In an exemplary embodiment, the motion encoding gradients are applied at different phases of the vibration signal and a corresponding phase correction is applied to each motion encoding by the respective one of the motion encoding gradients. If the interleaved motion encoding gradients are applied at different time points, i.e. different phases, a respective phase correction has to be applied to each motion encoding. In an exemplary embodiment, the phase correction is performed after temporal Fourier transform followed by an optional subtraction of a reference background phase.

In an exemplary embodiment, Magnetic Resonance Imaging reconstruction is performed based on signals obtained from the applied motion encoding gradients. Thus, standard MRI reconstruction techniques can be used for MRE.

According to an exemplary embodiment, a result from the reconstruction is used for evaluating stiffness and/or viscosity of an object being examined by the MRE. Such evaluation can be performed by a user or automatically (e.g. by image processing).

In an exemplary embodiment, the natural number of vibration periods within one sampling period is equal to one, two or three. This means that the motion encoding gradients and the reference scan have to be provided/performed within one, two or three vibration periods plus the respective fixed delay. This further means that the sampling period and the vibration period are of the same order of magnitude. Otherwise, if a comparatively high vibration frequency is necessary, the number of vibration periods within one sampling period may also be higher than three.

In an exemplary embodiment, the fixed delay corresponds to one third, one quarter or one fifth of the vibration period. This means that the phase shift between vibration signal and sampling period is zero after three, four or five sampling periods. This also means that the vibration period is sampled three, four or five times. Such sampling rate is sufficient for obtaining meaningful scans.

According to an exemplary embodiment, the sampling period may comprise four time slots, one of the four time slots for each of the three motion encoding gradients and one time slot for the reference scan as already indicated above. Thus, the gradients are not interleaved but applied successively one after another. After the application of the three motion encoding gradients the reference scan is performed. Consequently, a reference scan is performed in each sampling period. Thus, the MRE data acquisition is more reliable.

In an exemplary embodiment of the before mentioned scheme, the four time slots are the same size. Thus, e.g. an analog-digital converter can be used for clocking the MRE scans.

According to an exemplary embodiment, the three motion encoding gradients represent gradients in three orthogonal space directions. Specifically, there may be applied an x-gradient, a y-gradient and a z-gradient successively. Thus, a total 3D volume can be stimulated by the motion encoding gradients.

In an exemplary embodiment, no motion encoding gradient is applied during the reference scan. As explained above, the reference scan shall provide reference data for standardizing the other measurements. Thus, a large number of scans can be related to each other.

In an exemplary embodiment, the above described method is repeated as k-space point acquisition step for each k-space point of a k-space. In other words, the specific acquisition scheme with the sampling period aligned with a vibrational signal as defined above is applied for each k-space point of the k-space. At the end of the acquisition of the complete k-space the data are back transformed in order to obtain the MRE signal of a sample.

According to an exemplary embodiment of the inventive method, the k-space point acquisition steps are synchronized with a vibrational signal. The synchronization is realized by the fact that a multiple of the fixed delay corresponds to the vibrational period and the multiple itself is equal to the sampling number for sampling one vibrational period.

In an exemplary embodiment, the vibrational signal has a frequency in the range of 20 Hz to 200 Hz. For example, the frequency of the vibrational signal is 60 Hz. Such vibration frequency causes proper share waves in samples like human tissue in order to gain meaningful MRE signals.

The above object is also solved by a Magnetic Resonance Elastography device that may comprise signal generating means for providing a periodical vibration signal for exciting mechanical vibrations (within an object to be examined) with a vibration period, sampling means for sampling the vibration signal with a sampling period (corresponding to one wave phase), the sampling period corresponding to a natural number including zero of vibration periods plus a fixed delay, wherein the fixed delay multiplied with a sampling number is equal to the vibration period, the sampling number being a natural number greater than two, and gradient application means for applying in each sampling period: one motion encoding gradient each in three motion encoding scans and no motion encoding gradient in one reference scan.

The advantages and further developments of the above-described method also apply to the Magnetic Resonance Elastography device analogously. Thus, the mentioned method steps can be regarded as functional features of the device.

Furthermore, according to the present disclosure there is provided a computer program product comprising instructions which, when the program is executed by a Magnetic Resonance Elastography device as described above, cause the Magnetic Resonance Elastography device to carry out one of the above defined methods. Additionally, a computer-readable medium comprising instructions which, when executed by a Magnetic Resonance Elastography device as mentioned above, cause the Magnetic Resonance Elastography device to carry out one of the above defined methods.

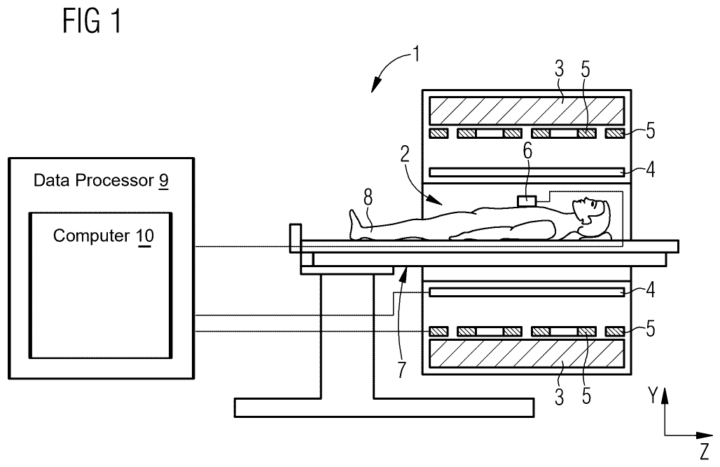

FIG. 1 shows a schematic representation of an exemplary implementation of an MRE-system 1 according to the disclosure.

The MRE-system may comprise a magnet unit with an imaging region 2, for example within a patient tunnel for placing an object 8, in particular a patient, to be imaged. The magnet unit may comprise a field magnet 3 that generates a main magnet field for aligning nuclear spins of the object, in particular within the imaging region 2. The imaging region 2 is characterized by a very homogeneous static main magnet field, the homogeneity relating, in particular, to the magnetic field strength. The field magnet 3 may, for example, be a superconducting magnet capable of providing magnetic fields with a magnetic flux density in the order of several Tesla, in particular in the order of 7 T or more. A patient table 7 may be movable within the patient tunnel.

Furthermore, the magnet unit may comprise a gradient coil arrangement 5 with several gradient coils that are designed to superimpose location-dependent magnetic fields in the three spatial directions on the static main magnet field for spatial differentiation and, in particular, slice selection. The gradient coils of the gradient coil arrangement 5 may, for example, be designed as coils of normal conducting wires, which may, for example, generate mutually orthogonal fields or field gradients in the recording region.

The MRE-system 1, in particular the magnet unit, may comprise a transmission coil arrangement, which contains one or more RF-coils 4. It is noted that the one or more RF-coils 4 of the transmission coil arrangement may, depending on the specific implementation or application, also be used as receiving coils. Optionally, the MRE-system 1 may also comprise one or more local coils (not shown in FIG. 1), which may be arranged in the immediate vicinity of the object 8, for example on the object 8 or in the patient table 7. The local coils may serve as receiving coils and/or transmission coils.

Furthermore, the MRE-system may comprise a (pulse) wave generator 6 for exciting shear waves within the object/patient to be examined.

The MRE-system 1 may also comprise a data processing apparatus 9 including at least one computing unit (computer) 10. The at least one computer 10 may be configured to carry out a computer-implemented method for controlling the MRE-system according to a specific timing scheme. Particularly, the time control of the wave generator 6 is synchronized with the MR imaging. As a result of the computer-implemented method, the at least one computer 10 controls the of the MRE-system so that the acquisition is performed in a very efficient way. The computer 10 may be referred to as a controller in one or more aspects. In an exemplary embodiment, the data processor 9 may include processing circuitry that is configured to perform one or more functions and/or operations of the data processor 9. One or more components of the data processor 9, such as the computer 10, may include processing circuitry that is configured to perform one or more functions and/or operations of the component(s) (e.g., the computer 10). Similarly, one or more components of the computer 10 may include processing circuitry that is configured to perform one or more respective functions and/or operations of the component(s).

In response to the excitation RF-pulses, the at least one computer 10 receives corresponding NMR-signals from the receiving coils and may generate respective MR-images of the object 8 depending on those signals.

In particular, the at least one computer 10 may comprise a readout controller, which is connected to the at least one RF-coil 4 and/or the local coil. Depending on the detected MR-signals, the readout controller, which may comprise an analogue-to-digital converter, ADC, may generate corresponding MR-data, in particular in k-space.

The at least one computer 10 may evaluate the MR-data and, for example, carry out a two-dimensional or three-dimensional image reconstruction based on the MR-data. The at least one computer 10 may also comprise a sending controller, which is connected to and controls the RF-coil(s) 4 and/or the local coil to emit the excitation RF-pulses and, for example refocusing RF-pulses and other RF-pulses. The at least one computer 10 may comprise gradient controller, which is connected to and controls the gradient coil arrangement 5 to apply, for example, slice selecting gradients, gradients for frequency and/or phase encoding, defocusing gradients and/or readout gradients and so forth. Additionally, the at least one computer 10 may comprise a wave generation controller adapted to control the wave generator.

It is noted that the described structure of the MRE-system 1 is a non-limiting example only. The different required tasks and functions may also be distributed differently and/or to different units in other applications.

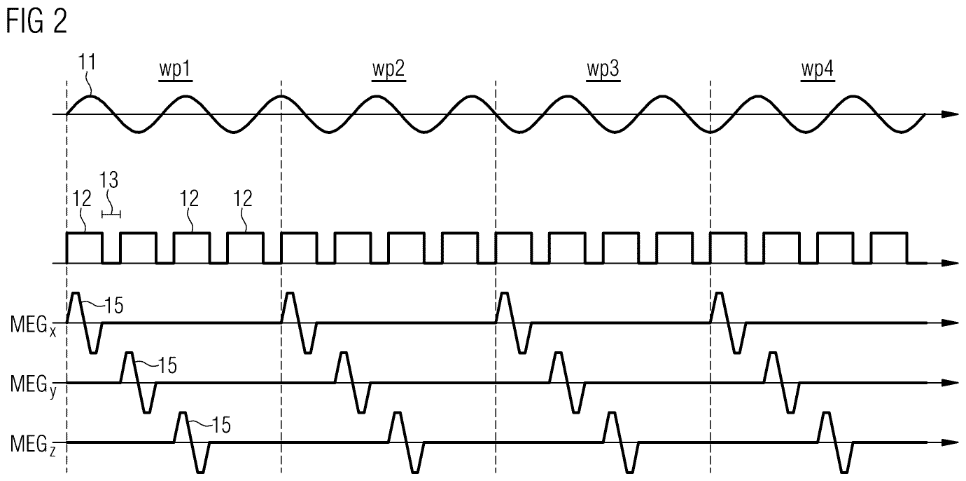

The design of a proposed 3D MRE sequence with 3D slab-selective phase-contrast spoiled gradient echo acquisition is shown in FIG. 2.

The acquisition of MRE data is performed in the k-space. For each k-space point of the k-space the acquisition scheme of FIG. 2 is performed. This acquisition scheme may comprise an outer loop and an inner loop. The outer loop results from the sampling of vibration wave 11 (shear wave). In the case of FIG. 2 the vibration wave 11 is sampled four times in one period. Thus, four waves phases wp1, wp2, wp3 and wp4 result. In other words, the vibration wave 11 is sampled each quarter wave length. The frequency of the vibration wave 11 may be 60 Hz. However, the proposed technique is not limited to this frequency. Any other appropriate frequency may be used.

In the preset example the 3D acquisition of one sampling point takes e.g. 37.5 ms which corresponds to two and a quarter wave length of a 60 Hz vibration wave.

Within each wave phase wp1, wp2, wp3 and wp4 an inner acquisition loop is performed. The inner acquisition loop may comprise four time slots 12. Each time slot 12 is separated from the next time slot 12 by a time delay 13. Specifically, in this example each wave phase wp1, wp2, wp3 and wp4 comprises four time slots 12 each followed by a time delay 13. Thus, the timing signal including the time slots 12 and the time delays 13 is a periodical signal with phase Phi=0 at each sampling point of the vibration wave 11.

Each inner acquisition loop includes three motion encodings and a reference scan without motion encoding. Specifically, a motion encoding gradient MEGₓ is applied in the first time slot 12, a second motion encoding gradient MEG_{y} is applied in the second time slot 12 and a third motion encoding gradient MEG_{z} is applied in the third time slot 12. No motion encoding gradient is applied during the fourth time slot 12, which is used for the reference scan. The constant delay at the end of each imaging readout shifts the acquisition to the successive wave offset, similar to the MRE sequence of Ristretto et al. mentioned in the introductory part of this document. As a consequence, the total acquisition time is minimized.

This acquisition scheme of FIG. 2 is repeated for all wave phases wp1, wp2, wp3 and wp4 until all wave offsets are acquired. Then the next k-space point is acquired in a similar manner. Advantageously each k-space point acquisition is synchronized with the mechanical vibration. An important aspect is that for each motion encoding step, the design of the sequence allows to measure all k-space points in the 2D phase-encoding plane at the same wave motion state, thereby avoiding motion related ghosting artifacts.

The sequence can be combined with any 2D phase encoding acceleration technique.

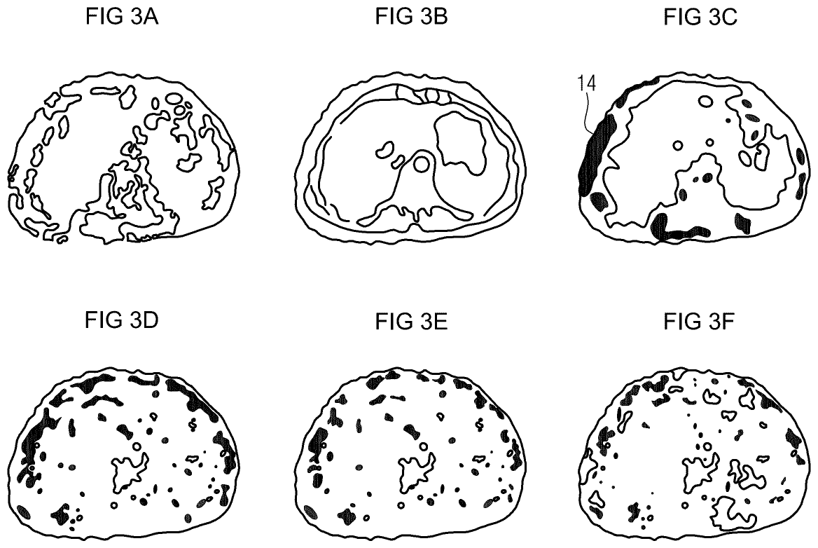

The proposed sequence cab be implemented on a 3 T system to realize a 3D slab-selective (e.g. liver) MRE measurement in a single breath-hold. Experiments were performed with 60 Hz actuation frequency with four wave-phase offsets and a four-point unbalanced motion encoding scheme. A breath-hold duration of 22 seconds was necessary. The proposed sequence was validated for a liver acquisition in one healthy subject as shown in FIG. 3.

The obtained phase images may be unwrapped using the minimum cost flow technique as stated by Constantini. The n-th motion encoding step (n=1 . . . 3) in the proposed sequence is acquired at a different time with respect to the 0-th motion encoding step and therefore accumulates a phase φₙ=2πₙ φ_{inc} where φ_{inc}=(N_{Tvib})/(4 N_{wp}), where N_{Tvib} is the number of vibration periods required for the acquisition of all wave offsets and motion encodings for one k-space point and N_{wp} is the number of acquired wave offsets. Therefore, to obtain the 3D displacement field, a phase correction scheme is applied to each motion encoding after the temporal Fourier transform followed by the subtraction of the reference background phase. A 3D Gaussian filter of width σ=2 pixels and a support of 3×3×3 pixels was applied to the displacement field. MRE reconstruction was performed using the curl operator to remove the compressional wave followed by a direct inversion of the complex wave equation as proposed by Sinkus et. al. The following parameters were retrieved: the magnitude of the complex-valued shear modulus (|G*|[kPa]), the shear wave speed (Cs [m/s]), and the loss modulus (G″ [kPa]).

Respective results are shown in FIG. 3. Picture A of FIG. 3 is the calculated curl of the wave field in the Z direction of the object (here liver). This can represent the propagation of the shear wave in the liver/object. Picture B of FIG. 3 represents the magnitude of the reference scan without motion encoding. The picture corresponds to a usual MRT image. Picture C of FIG. 3 shows the total wave amplitude. The black regions 14 reflect higher amplitude of vibrations. Pictures D to F of FIG. 3 show viscoelastic parameters to explain the behavior/state of the tissue. Picture D represents the shear wave speed Cs. Cs describes how stiff tissue is. Fibrotic liver would be stiff and would be recognizable by elevated Cs. Picture E of FIG. 3 shows the magnitude of the complex valued shear modulus |G*|, which is correlated with the speed Cs. |G*| displays how stiff a tissue is (e.g. fibrotic liver is stiff). Picture F of FIG. 3 shows the loss modulus G″. It is describes the viscous behavior of the tissue (for example honey is very viscous) and research argues that inflamed liver is more viscus.

Compared to previously published 3D MRE techniques, the above embodiments allow a more efficient scheme than the method of van Schelt that is based on wave offset interleaving that requires a substantial increase in TR and an acquisition of five wave offsets instead of four. The present technique may also be based on a cartesian trajectory, which is less sensitive to eddy currents and off-resonance effects than a 3D fast MRE sequence based on a spiral trajectory.

To enable those skilled in the art to better understand the solution of the present disclosure, the technical solution in the embodiments of the present disclosure is described clearly and completely below in conjunction with the drawings in the embodiments of the present disclosure. Obviously, the embodiments described are only some, not all, of the embodiments of the present disclosure. All other embodiments obtained by those skilled in the art on the basis of the embodiments in the present disclosure without any creative effort should fall within the scope of protection of the present disclosure.

It should be noted that the terms “first”, “second”, etc. in the description, claims and abovementioned drawings of the present disclosure are used to distinguish between similar objects, but not necessarily used to describe a specific order or sequence. It should be understood that data used in this way can be interchanged as appropriate so that the embodiments of the present disclosure described here can be implemented in an order other than those shown or described here. In addition, the terms “comprise” and “have” and any variants thereof are intended to cover non-exclusive inclusion. For example, a process, method, system, product or equipment comprising a series of steps or modules or units is not necessarily limited to those steps or modules or units which are clearly listed, but may comprise other steps or modules or units which are not clearly listed or are intrinsic to such processes, methods, products or equipment.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes, and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact results from computing devices, processors, controllers, or other devices executing the firmware, software, routines, instructions, etc. Further, any of the implementation variations may be carried out by a general-purpose computer.

The various components described herein may be referred to as “modules,” “units,” or “devices.” Such components may be implemented via any suitable combination of hardware and/or software components as applicable and/or known to achieve their intended respective functionality. This may include mechanical and/or electrical components, processors, processing circuitry, or other suitable hardware components, in addition to or instead of those discussed herein. Such components may be configured to operate independently, or configured to execute instructions or computer programs that are stored on a suitable computer-readable medium. Regardless of the particular implementation, such modules, units, or devices, as applicable and relevant, may alternatively be referred to herein as “circuitry,” “controllers,” “processors,” or “processing circuitry,” or alternatively as noted herein.

For the purposes of this discussion, the term “processing circuitry” shall be understood to be circuit(s) or processor(s), or a combination thereof. A circuit includes an analog circuit, a digital circuit, data processing circuit, other structural electronic hardware, or a combination thereof. A processor includes a microprocessor, a digital signal processor (DSP), central processor (CPU), application-specific instruction set processor (ASIP), graphics and/or image processor, multi-core processor, or other hardware processor. The processor may be “hard-coded” with instructions to perform corresponding function(s) according to aspects described herein. Alternatively, the processor may access an internal and/or external memory to retrieve instructions stored in the memory, which when executed by the processor, perform the corresponding function(s) associated with the processor, and/or one or more functions and/or operations related to the operation of a component having the processor included therein.

In one or more of the exemplary embodiments described herein, the memory is any well-known volatile and/or non-volatile memory, including, for example, read-only memory (ROM), random access memory (RAM), flash memory, a magnetic storage media, an optical disc, erasable programmable read only memory (EPROM), and programmable read only memory (PROM). The memory can be non-removable, removable, or a combination of both.

1. A method of performing Magnetic Resonance Elastography (MRE), the method comprising:
providing a periodical vibration signal to excite mechanical vibrations with a vibration period;
sampling the vibration signal with a sampling period corresponding to a natural number including zero of vibration periods plus a fixed delay, wherein a product of the fixed delay and a sampling number is equal to the vibration period, the sampling number being a natural number greater than two; and
applying three motion encoding gradients in each sampling period for magnetic resonance acquisition.

2. The method according to claim 1, wherein the motion encoding gradients are part of a three-dimensional (3D) slab-selective Magnetic Resonance Imaging sequence.

3. The method according to claim 1, wherein the motion encoding gradients are applied at different phases of the vibration signal and a corresponding phase correction is applied to each motion encoding by the respective one of the motion encoding gradients.

4. The method according to claim 1, further comprising preforming Magnetic Resonance Imaging reconstruction based on signals obtained from the applied motion encoding gradients.

5. The method according to claim 4, further comprising using a result from the reconstruction to evaluate stiffness and/or viscosity of an object being examined by the MRE.

6. The method according to claim 1, wherein the natural number of vibration periods within one sampling period is equal to one, two, or three.

7. The method according to claim 1, wherein the fixed delay corresponds to one third, one quarter, or one fifth of the vibration period.

8. The method according to claim 1, wherein the sampling period comprises four time slots, three of the four time slots for the three motion encoding gradients, respectively, and a remaining one of the four time slots for a reference scan.

9. The method according to claim 8, wherein the four time slots are a same size.

10. The method according to claim 1, wherein the three motion encoding gradients represent gradients in three orthogonal space directions.

11. The method according to claim 1, wherein no motion encoding gradient is applied during the reference scan.

12. The method according to claim 1, wherein the method is repeated as k-space point acquisition step for each k-space point of a k-space.

13. The method according to claim 12, wherein the k-space point acquisition steps are synchronized with the vibration signal.

14. A non-transitory computer-readable storage medium with an executable program stored thereon, that when executed, instructs a processor to perform the method of claim 1.

15. A Magnetic Resonance Elastography (MRE) device comprising:
a signal generator configured to provide a periodical vibration signal to excite mechanical vibrations with a vibration period;
a sampler configured to sample the vibration signal with a sampling period corresponding to a natural number including zero of vibration periods plus a fixed time delay, wherein a product of the fixed time delay and a sampling number is equal to the vibration period, the sampling number being a natural number greater than two; and
a gradient applicator configured to apply, in each sampling period, one motion encoding gradient each in three motion encoding scans and no motion encoding gradient in one reference scan.

16. A Magnetic Resonance Elastography (MRE) device comprising:
one or more processors; and
memory storing instructions that, when executed by the one or more processors, cause the MRE device to:
provide a periodical vibration signal to excite mechanical vibrations with a vibration period;
sample the vibration signal with a sampling period corresponding to a natural number including zero of vibration periods plus a fixed time delay, wherein a product of the fixed time delay and a sampling number is equal to the vibration period, the sampling number being a natural number greater than two; and
apply, in each sampling period, one motion encoding gradient each in three motion encoding scans and no motion encoding gradient in one reference scan.
